SEQUENCE LISTING (177 sequences)

SEQ ID NO: 1 - DNA, 20 bp, Homo sapiens
aaataatcccatccaactac

SEQ ID NO: 2 - DNA, 20 bp, Homo sapiens
gtagttggatgggattattt

SEQ ID NO: 3 - DNA, 21 bp, Homo sapiens
aaataatcccatccaactacg

SEQ ID NO: 4 - DNA, 21 bp, Homo sapiens
cgtagttggatgggattattt

SEQ ID NO: 5 - DNA, 19 bp, Homo sapiens
aataatcccatccaactac

SEQ ID NO: 6 - DNA, 19 bp, Homo sapiens
gtagttggatgggattatt

SEQ ID NO: 7 - DNA, 20 bp, Homo sapiens
aataatcccatccaactacg

SEQ ID NO: 8 - DNA, 20 bp, Homo sapiens
cgtagttggatgggattatt

SEQ ID NO: 9 - DNA, 19 bp, Homo sapiens
ataatcccatccaactacg

SEQ ID NO: 10 - DNA, 19 bp, Homo sapiens
cgtagttggatgggattat

SEQ ID NO: 11 - DNA, 20 bp, Homo sapiens
raaataatcccatccaacta

SEQ ID NO: 12 - DNA, 21 bp, Homo sapiens
craaataatcccatccaacta

SEQ ID NO: 13 - DNA, 21 bp, Homo sapiens
tagttggatgggattatttyg

SEQ ID NO: 14 - DNA, 19 bp, Homo sapiens
raaataatcccatccaact

SEQ ID NO: 15 - DNA, 19 bp, Homo sapiens
agttggatgggattattty

SEQ ID NO: 16 - DNA, 20 bp, Homo sapiens
craaataatcccatccaact

SEQ ID NO: 17 - DNA, 20 bp, Homo sapiens
agttggatgggattatttyg

SEQ ID NO: 18 - DNA, 19 bp, Homo sapiens
craaataatcccatccaac

SEQ ID NO: 19 - DNA, 19 bp, Homo sapiens
gttggatgggattatttyg

SEQ ID NO: 20 - DNA, 20 bp, Homo sapiens
ccaccttcgaaatccgaaat

SEQ ID NO: 21 - DNA, 20 bp, Homo sapiens
atttcggatttcgaaggtgg

SEQ ID NO: 22 - DNA, 21 bp, Homo sapiens
atccgaaataatcccatccaa

SEQ ID NO: 23 - DNA, 21 bp, Homo sapiens
ttggatgggattatttcggat

SEQ ID NO: 24 - DNA, 22 bp, Homo sapiens
gatttcgaaggtgggtgttggg

SEQ ID NO: 25 - DNA, 22 bp, Homo sapiens
cccaacacccaccttcgaaatc

SEQ ID NO: 26 - DNA, 21 bp, Homo sapiens
tcgtcgttgtttttcgcgcga

SEQ ID NO: 27 - DNA, 21 bp, Homo sapiens
tcgcgcgaaaaacaacgacga

SEQ ID NO: 28 - DNA, 25 bp, Homo sapiens
cataataactaataaacaacraatc

SEQ ID NO: 29 - DNA, 25 bp, Homo sapiens
gattygttgtttattagttattatg

SEQ ID NO: 30 - DNA, 24 bp, Homo sapiens
gattygttgtttattagttattat

SEQ ID NO: 31 - DNA, 24 bp, Homo sapiens
ataataactaataaacaacraatc

SEQ ID NO: 32 - DNA, 21 bp, Homo sapiens
gttgtttattagttattatgt

SEQ ID NO: 33 - DNA, 21 bp, Homo sapiens
acataataactaataaacaac

SEQ ID NO: 34 - DNA, 16 bp, Homo sapiens
gtagggttcgggtttc

SEQ ID NO: 35 - DNA, 16 bp, Homo sapiens
gaaacccgaaccctac

SEQ ID NO: 36 - DNA, 16 bp, Homo sapiens
ttcgtcgctgtttttc

SEQ ID NO: 37 - DNA, 16 bp, Homo sapiens
gaaaaacagcgacgaa

SEQ ID NO: 38 - DNA, 20 bp, Homo sapiens
gcgcgattcgttgtttatta

SEQ ID NO: 39 - DNA, 20 bp, Homo sapiens
taataaacaacgaatcgcgc

SEQ ID NO: 40 - DNA, 17 bp, Homo sapiens
tagggttcgggtttcgt

SEQ ID NO: 41 - DNA, 17 bp, Homo sapiens
acgaaacccgaacccta

SEQ ID NO: 42 - DNA, 16 bp, Homo sapiens
gtagggttcgggttty

SEQ ID NO: 43 - DNA, 16 bp, Homo sapiens
raaacccgaaccctac

SEQ ID NO: 44 - DNA, 16 bp, Homo sapiens
ttygtygctgttttty

SEQ ID NO: 45 - DNA, 16 bp, Homo sapiens
raaaaacagcracraa

SEQ ID NO: 46 - DNA, 20 bp, Homo sapiens
gygygattygttgtttatta

SEQ ID NO: 47 - DNA, 20 bp, Homo sapiens
taataaacaacraatcrcrc

SEQ ID NO: 48 - DNA, 17 bp, Homo sapiens
tagggttygggtttygt

SEQ ID NO: 49 - DNA, 17 bp, Homo sapiens
acraaacccraacccta

SEQ ID NO: 50 - DNA, 26 bp, Homo sapiens
ygattygttgtttattagttattatg

SEQ ID NO: 51 - DNA, 26 bp, Homo sapiens
cataataactaataaacaacraatcr

SEQ ID NO: 52 - DNA, 24 bp, Homo sapiens
ttggattttgtggttaatgtgtag

SEQ ID NO: 53 - DNA, 26 bp, Homo sapiens
tgttggattttgtggttaatgtgtag

SEQ ID NO: 54 - DNA, 21 bp, Homo sapiens
gttattatgttggattttgtg

SEQ ID NO: 55 - DNA, 32 bp, Homo sapiens
gttattatgttggattttgtggttaatgtgta

SEQ ID NO: 56 - DNA, 31 bp, Homo sapiens
gttattatgttggattttgtggttaatgtgt

SEQ ID NO: 57 - DNA, 30 bp, Homo sapiens
gttattatgttggattttgtggttaatgtg

SEQ ID NO: 58 - DNA, 23 bp, Homo sapiens
gttattatgttggattttgtggt

SEQ ID NO: 59 - DNA, 37 bp, Homo sapiens
gttattatgttggattttgtggttaatgtgtagaaaa

SEQ ID NO: 60 - DNA, 32 bp, Homo sapiens
tgtttattagttattatgttggattttgtggt

SEQ ID NO: 61 - DNA, 35 bp, Homo sapiens
ttattatgttggattttgtggttaatgtgtagaaa

SEQ ID NO: 62 - DNA, 30 bp, Homo sapiens
tttattagttattatgttggattttgtggt

SEQ ID NO: 63 - DNA, 29 bp, Homo sapiens
ttattagttattatgttggattttgtggt

SEQ ID NO: 64 - DNA, 28 bp, Homo sapiens
tattagttattatgttggattttgtggt

SEQ ID NO: 65 - DNA, 27 bp, Homo sapiens
attagttattatgttggattttgtggt

SEQ ID NO: 66 - DNA, 26 bp, Homo sapiens
ttagttattatgttggattttgtggt

SEQ ID NO: 67 - DNA, 25 bp, Homo sapiens
tagttattatgttggattttgtggt

SEQ ID NO: 68 - DNA, 18 bp, Homo sapiens
ttcgcggttaacgcgtag

SEQ ID NO: 69 - DNA, 18 bp, Homo sapiens
ctacgcgttaaccgcgaa

SEQ ID NO: 70 - DNA, 18 bp, Homo sapiens
atttcgcggttaacgcgt

SEQ ID NO: 71 - DNA, 18 bp, Homo sapiens
acgcgttaaccgcgaaat

SEQ ID NO: 72 - DNA, 18 bp, Homo sapiens
gttaaccgcgaaatccga

SEQ ID NO: 73 - DNA, 18 bp, Homo sapiens
tcggatttcgcggttaac

SEQ ID NO: 74 - DNA, 19 bp, Homo sapiens
cgttaaccgcgaaatccga

SEQ ID NO: 75 - DNA, 19 bp, Homo sapiens
tcggatttcgcggttaacg

SEQ ID NO: 76 - DNA, 20 bp, Homo sapiens
gcgttaaccgcgaaatccga

SEQ ID NO: 77 - DNA, 20 bp, Homo sapiens
tcggatttcgcggttaacgc

SEQ ID NO: 78 - DNA, 21 bp, Homo sapiens
cgcgttaaccgcgaaatccga

SEQ ID NO: 79 - DNA, 18 bp, Homo sapiens
cgttaaccgcgaaatccg

SEQ ID NO: 80 - DNA, 20 bp, Homo sapiens
gcgttaaccgcgaaatccga

SEQ ID NO: 81 - DNA, 21 bp, Homo sapiens
cgcgttaaccgcgaaatccga

SEQ ID NO: 82 - DNA, 18 bp, Homo sapiens
gtcggatttcgcggttaa

SEQ ID NO: 83 - DNA, 19 bp, Homo sapiens
gtcggatttcgcggttaac

SEQ ID NO: 84 - DNA, 20 bp, Homo sapiens
gtcggatttcgcggttaacg

SEQ ID NO: 85 - DNA, 21 bp, Homo sapiens
gtcggatttcgcggttaacgc

SEQ ID NO: 86 - DNA, 22 bp, Homo sapiens
gtcggatttcgcggttaacgcg

SEQ ID NO: 87 - DNA, 18 bp, Homo sapiens
tcggatttcgcggttaac

SEQ ID NO: 88 - DNA, 18 bp, Homo sapiens
cggatttcgcggttaacg

SEQ ID NO: 89 - DNA, 19 bp, Homo sapiens
cggatttcgcggttaacgc

SEQ ID NO: 90 - DNA, 20 bp, Homo sapiens
cggatttcgcggttaacgcg

SEQ ID NO: 91 - DNA, 22 bp, Homo sapiens
agtaagaagagttttagagagt

SEQ ID NO: 92 - DNA, 22 bp, Homo sapiens
actctctaaaactcttcttact

SEQ ID NO: 93 - DNA, 21 bp, Homo sapiens
gaagagttttagagagtagtt

SEQ ID NO: 94 - DNA, 21 bp, Homo sapiens
aactactctctaaaactcttc

SEQ ID NO: 95 - DNA, 18 bp, Homo sapiens
cgcgttttcggggcgtag

SEQ ID NO: 96 - DNA, 18 bp, Homo sapiens
ctacgccccgaaaacgcg

SEQ ID NO: 97 - DNA, 18 bp, Homo sapiens
ggaggaagcgagygtttt

SEQ ID NO: 98 - DNA, 18 bp, Homo sapiens
aaaacrctcgcttcctcc

SEQ ID NO: 99 - DNA, 21 bp, Homo sapiens
tagaaattaataagtgagagg

SEQ ID NO: 100 - DNA, 21 bp, Homo sapiens
cctctcacttattaatttcta

SEQ ID NO: 101 - DNA, 17 bp, Homo sapiens
ttcggggcgtagttgcg

SEQ ID NO: 102 - DNA, 17 bp, Homo sapiens
cgcaactacgccccgaa

SEQ ID NO: 103 - DNA, 21 bp, Homo sapiens
gcgtaggaggaggaagcgagc

SEQ ID NO: 104 - DNA, 21 bp, Homo sapiens
gctcgcttcctcctcctacgc

SEQ ID NO: 105 - DNA, 21 bp, Homo sapiens
gygtaggaggaggaagcgagc

SEQ ID NO: 106 - DNA, 21 bp, Homo sapiens
gctcgcttcctcctcctacrc

SEQ ID NO: 107 - DNA, 21 bp, Homo sapiens
gygtaggaggaggaagygagc

SEQ ID NO: 108 - DNA, 21 bp, Homo sapiens
gctcrcttcctcctcctacrc

SEQ ID NO: 109 - DNA, 21 bp, Homo sapiens
gygtaggaggaggaagygagy

SEQ ID NO: 110 - DNA, 21 bp, Homo sapiens
rctcrcttcctcctcctacrc

SEQ ID NO: 111 - DNA, 18 bp, Homo sapiens
cgtaggaggaggaagcga

SEQ ID NO: 112 - DNA, 18 bp, Homo sapiens
tcgcttcctcctcctacg

SEQ ID NO: 113 - DNA, 18 bp, Homo sapiens
ygtaggaggaggaagyga

SEQ ID NO: 114 - DNA, 18 bp, Homo sapiens
tcrcttcctcctcctacr

SEQ ID NO: 115 - DNA, 18 bp, Homo sapiens
aataagtgagagggcgtc

SEQ ID NO: 116 - DNA, 18 bp, Homo sapiens
gacgccctctcacttatt

SEQ ID NO: 117 - DNA, 19 bp, Homo sapiens
ttaataagtgagagggtcg

SEQ ID NO: 118 - DNA, 19 bp, Homo sapiens
gtttcggattcgtgtgcgc

SEQ ID NO: 119 - DNA, 19 bp, Homo sapiens
gcgcacacgaatccgaaac

SEQ ID NO: 120 - DNA, 20 bp, Homo sapiens
cggattcgtgtgcgcgggtt

SEQ ID NO: 121 - DNA, 20 bp, Homo sapiens
aacccgcgcacacgaatccg

SEQ ID NO: 122 - DNA, 23 bp, Homo sapiens
gcggtattttgtttcggattcgt

SEQ ID NO: 123 - DNA, 23 bp, Homo sapiens
acgaatccgaaacaaaataccgc

SEQ ID NO: 124 - DNA, 19 bp, Homo sapiens
cgtaatcgttgcggtattt

SEQ ID NO: 125 - DNA, 19 bp, Homo sapiens
aaataccgcaacgattacg

SEQ ID NO: 126 - DNA, 20 bp, Homo sapiens
ttcgagttttcgagtttgag

SEQ ID NO: 127 - DNA, 20 bp, Homo sapiens
ctcaaactcgaaaactcgaa

SEQ ID NO: 128 - DNA, 19 bp, Homo sapiens
cgtaatcgttgcrgtattt

SEQ ID NO: 129 - DNA, 19 bp, Homo sapiens
aaatacygcaacgattacg

SEQ ID NO: 130 - DNA, 19 bp, Homo sapiens
cgtaatcrttgcrgtattt

SEQ ID NO: 131 - DNA, 19 bp, Homo sapiens
aaatacygcaaygattacg

SEQ ID NO: 132 - DNA, 19 bp, Homo sapiens
crtaatcrttgcrgtattt

SEQ ID NO: 133 - DNA, 19 bp, Homo sapiens
aaatacygcaaygattayg

SEQ ID NO: 134 - DNA, 20 bp, Homo sapiens
ttcgagttttcragtttgag

SEQ ID NO: 135 - DNA, 20 bp, Homo sapiens
ctcaaactygaaaactcgaa

SEQ ID NO: 136 - DNA, 20 bp, Homo sapiens
ttcragttttcragtttgag

SEQ ID NO: 137 - DNA, 20 bp, Homo sapiens
ctcaaactygaaaactygaa

SEQ ID NO: 138 - DNA, 22 bp, Homo sapiens
cgagttcgagttttcgagtttg

SEQ ID NO: 139 - DNA, 22 bp, Homo sapiens
caaactcgaaaactcgaactcg

SEQ ID NO: 140 - DNA, 21 bp, Homo sapiens
cgagttcgagttttcgagttt

SEQ ID NO: 141 - DNA, 21 bp, Homo sapiens
aaactcgaaaactcgaactcg

SEQ ID NO: 142 - DNA, 20 bp, Homo sapiens
cgagttcgagttttcgagtt

SEQ ID NO: 143 - DNA, 20 bp, Homo sapiens
aactcgaaaactcgaactcg

SEQ ID NO: 144 - DNA, 24 bp, Homo sapiens
gtaggtgtaggaggaggaagtgag

SEQ ID NO: 145 - DNA, 24 bp, Homo sapiens
ctcacttcctcctcctacacctac

SEQ ID NO: 146 - DNA, 22 bp, Homo sapiens
tttggggtgtagttgtgggtgg

SEQ ID NO: 147 - DNA, 22 bp, Homo sapiens
ccacccacaactacaccccaaa

SEQ ID NO: 148 - DNA, 22 bp, Homo sapiens
tgagtttgagtttttgagtttg

SEQ ID NO: 149 - DNA, 22 bp, Homo sapiens
caaactcaaaaactcaaactca

SEQ ID NO: 150 - DNA, 20 bp, Homo sapiens
tttgagtttgagtttttgag

SEQ ID NO: 151 - DNA, 20 bp, Homo sapiens
ctcaaaaactcaaactcaaa

SEQ ID NO: 152 - DNA, 20 bp, Homo sapiens
ggcgtagttgcgggcggcgg

SEQ ID NO: 153 - DNA, 20 bp, Homo sapiens
ccgccgcccgcaactacgcc

SEQ ID NO: 154 - DNA, 25 bp, Homo sapiens
tgagtgtttttgagttttgagtttg

SEQ ID NO: 155 - DNA, 25 bp, Homo sapiens
aacttgaaaacttgaacttgaaact

SEQ ID NO: 156 - DNA, 33 bp, Homo sapiens
tgagtgtttttgagttttgagtttgagtttttg

SEQ ID NO: 157 - DNA, 23 bp, Homo sapiens
ggcgtaggaggaggaagcgagcg

SEQ ID NO: 158 - DNA, 23 bp, Homo sapiens
cgctcgcttcctcctcctacgcc

SEQ ID NO: 159 - DNA, 18 bp, Homo sapiens
ggcgtagttgcgggcggc

SEQ ID NO: 160 - DNA, 18 bp, Homo sapiens
gccgcccgcaactacgcc

SEQ ID NO: 161 - DNA, 17 bp, Homo sapiens
tagttgcgggcggcggg

SEQ ID NO: 162 - DNA, 17 bp, Homo sapiens
cccgccgcccgcaacta

SEQ ID NO: 163 - DNA, 22 bp, Homo sapiens
agttcgagttttcgagtttgag

SEQ ID NO: 164 - DNA, 25 bp, Homo sapiens
aactcgaaaactcgaactcgaaact

SEQ ID NO: 165 - DNA, 25 bp, Homo sapiens
agtttcgagttcgagttttcgagtt

SEQ ID NO: 166 - DNA, 22 bp, Homo sapiens
ctcaaactcgaaaactcgaact

SEQ ID NO: 167 - DNA, 19 bp, Homo sapiens
gcgtaggaggaggaagcga

SEQ ID NO: 168 - DNA, 20 bp, Homo sapiens
cgggcggcgggagtaggcgt

SEQ ID NO: 169 - DNA, 19 bp, Homo sapiens
tcgcttcctcctcctacgc

SEQ ID NO: 170 - DNA, 20 bp, Homo sapiens
acgcctactcccgccgcccg

SEQ ID NO: 171 - DNA, 20 bp, Homo sapiens
cgtaggaggaggaagcgagc

SEQ ID NO: 172 - DNA, 20 bp, Homo sapiens
gctcgcttcctcctcctacg

SEQ ID NO: 173 - DNA, 24 bp, Homo sapiens
cgagttcgagttttcgagtttgag

SEQ ID NO: 174 - DNA, 24 bp, Homo sapiens
ctcaaactcgaaaactcgaactcg

SEQ ID NO: 175 - DNA, 23 bp, Homo sapiens
gtgatggaggaggtttagtaagt

SEQ ID NO: 176 - DNA, 24 bp, Homo sapiens
ccaataaaacctactcctccctta

SEQ ID NO: 177 - DNA, 28 bp, Homo sapiens
ccaccacccaacacacaataacaaacac

(19) United States
(12) Patent Application Publication
(10) Pub. No.: US 2021/0087636 A1
(43) Pub. Date: Mar. 25, 2021

(54) PRIMER AND PROBE SET FOR DIAGNOSIS, DETECTION OR SCREENING OF COLORECTAL CANCER

(71) Applicant: SUZHOU VERSABIO TECHNOLOGIES, INC., Suzhou, Jiangsu (CN)

(72) Inventor: Guodong ZHAO, Suzhou, Jiangsu (CN)

(73) Assignee: SUZHOU VERSABIO TECHNOLOGIES, INC., Suzhou, Jiangsu (CN)

(21) Appl. No.: 16/982,577

(22) PCT Filed: Jan. 8, 2019
(86) PCT No.: PCT/CN2019/070750

§ 371 Date: Sep. 20, 2020

(30) Foreign Application Priority Data
Jun. 6, 2018 (CN) 201810587088.X

(51) Int. Cl. C12Q 1/6886; C12Q 1/6851

(52) U.S. Cl. CPC C12Q 1/6886; C12Q 1/6851

(57) ABSTRACT

Disclosed is a primer and probe set for diagnosis, detection or screening of colorectal cancer. A forward primer of SEPT9 is selected from any one of SEQ ID NOs: 1-25, a reverse primer of SEPT9 is selected from any one of SEQ ID NOs: 26-51, the blocker primer of SEPT9 is selected from any one of SEQ ID NOs: 52-67, and the probe of SEPT9 is selected from any one of SEQ ID NOs: 68-90. A forward primer of SDC2 is selected from any one of SEQ ID NOs: 91-117, a reverse primer of SDC2 is selected from any one of SEQ ID NOs: 118-143, the blocker primer of SDC2 is selected from any one of SEQ ID NOs: 144-156, and the probe of SDC2 is selected from any one of SEQ ID NOs: 157-174.

### FIELD

The present invention relates to the biomedical field, and relates to a primer and probe set for simultaneously detecting methylation levels of SEPT9 and SDC2 in a biological sample, for use in screening/detection/diagnosis of early-stage colorectal cancer.

### BACKGROUND

Colorectal cancer is one of the most common malignant tumors. Cancer Statistics in China, 2015 showed that colorectal cancer ranked the top five among cancer types both in men and women, and colorectal cancer has been increasing year by year in the past decade. The current initial treatment staging proportion of colorectal cancer is: 15% in stage I, 20%-30% in stage II, 30%-40% in stage III, and 20%-25% in stage IV. In terms of survival, the 5-year survival rate of patients in stage I may reach more than 90%, while the survival rate of patients in stage IV is merely slightly greater than 10%. Therefore, application of early screening for colorectal cancer to achieve early diagnosis and early treatment is of great significance to improve the survival rate of patients and thus improve their overall health. Colonoscopy has many shortcomings as the gold standard for colorectal cancer screening, particularly in developing countries such as China. This method is highly demanding for operators. Moreover, as an invasive examination method, patients have great pain without anesthesia, leading to a low acceptance rate. At present, the screening rate is less than 3%. As a result, the early detection rate of colorectal cancer in China is much lower than that in developed countries.

At present, there are many methods for detecting colorectal cancer, such as fecal occult blood test, colonoscopy, computed tomography (CT), or fecal DNA detection. All of the above methods have been used to some extent, but they also have certain defects. Colonoscopy is currently the most sensitive method in colorectal cancer screening. However, this method requires professional operators and is expensive with low acceptance rates among patients, and has certain defects in early-stage colorectal cancer screening. The fecal occult blood test is a rapid detection method and is widely applied in colorectal cancer screening, but due to its low specificity, there are a large number of false positives, and the detection results are susceptible to interference from diet and drugs. Other methods, such as CT, may only detect cancers in the middle and advanced stages, so they cannot be used for early screening. The detection results of fecal DNA screening technology are easily interfered by impurities in feces. Moreover, the current fecal DNA detection method is to screen multiple biomarkers simultaneously, and due to the extremely high detection costs, it is currently difficult to popularize in developing countries like China. Furthermore, complicated operation steps also make it difficult to popularize in hospitals.

Abnormal DNA methylation is closely related to occurrence and persistence of many diseases, and especially, the research results in recent years show that DNA methylation plays an important role in induction and persistence of cancers, making it a good biomarker for many cancers.

SEPT9 is a very effective DNA methylation marker of cancer. Studies have shown that SETP9 presents significant expression differences in colorectal cancer tissues and normal tissues. Syndecan-2 (SDC2) protein generally functions as an integral membrane protein, and it is known to participate in cell proliferation, migration, and intercellular interactions through extracellular matrix protein receptors. The SDC2 gene is expressed in mesenchymal cells rather than epithelial cells in normal colon tissue. Studies have shown that patients with colorectal cancer show abnormal methylation of SDC2 at high frequency.

### SUMMARY

To overcome the defects of the current early screening/detection/diagnosis method for colorectal cancer, the present invention provides a primer and probe set for simultaneously detecting methylation levels of SEPT9 and SDC2 genes in a biological sample. Early screening/detection/diagnosis of colorectal cancer is realized by extracting DNA from the biological sample, and detecting whether the biological sample contains methylated SEPT9 or SDC2 genes by using quantitative fluorescent PCR technology.

In order to solve the above technical problem, the present invention adopts the following technical solution: a primer and probe set for diagnosis, detection or screening of colorectal cancer includes amplification primers, a blocker primer, and a probe of SEPT9, and amplification primers, a blocker primer, and a probe of SDC2.

A forward primer of SEPT9 is selected from any one of SEQ ID NOs: 1-25, a reverse primer of SEPT9 is selected from any one of SEQ ID NOs: 26-51, the blocker primer of SEPT9 is selected from any one of SEQ ID NOs: 52-67, and the probe of SEPT9 is selected from any one of SEQ ID NOs: 68-90.

A forward primer of SDC2 is selected from any one of SEQ ID NOs: 91-117, a reverse primer of SDC2 is selected from any one of SEQ ID NOs: 118-143, the blocker primer of SDC2 is selected from any one of SEQ ID NOs: 144-156, and the probe of SDC2 is selected from any one of SEQ ID NOs: 157-174.

Further included is a reference gene primer probe set, where the reference gene is ACTB, a forward primer of ACTB is SEQ ID NO: 175, a reverse primer of ACTB is SEQ ID NO: 176, and a probe of ACTB is selected from SEQ ID NO: 177.

A detection object is a DNA sample obtained by extracting and purifying genomic DNA from a biological sample and then converting same using bisulfite, and carrying out re-purification (the sample serves as a DNA template of a PCR reaction system, and after the bisulfite conversion, a DNA template required for PCR is obtained through a conventional purification operation). The bisulfite is one or more of ammonium bisulfite, sodium bisulfite, and anhydrous sodium sulfate, and the concentration of the bisulfite is 8-10 M; the reaction conditions of the bisulfite conversion process are: DNA is added into a bisulfite solution of 8-10 M, and heated at 70-90° C. for 30-60 min.

Detection of SEPT9 methylation, SDC2 methylation, and ACTB is performed simultaneously in one quantitative fluorescent PCR reaction, or a quantitative fluorescent PCR reaction is performed separately for one site (SEPT9, SDC2 or ACTB).

The blocker primer of SEPT9 and/or the blocker primer of SDC2 are modified using one of a phosphate group, C3, C9, and C18 at 3′-ends.

The present invention further provides an amplification system including the primer and probe set, where the specific components of the amplification system are:

| Components | Final concentration | |
|---|---|---|
| PCR reaction buffer | 1 | X |
| DNA polymerase | 0.08-0.2 | U/μL |
| dNTPs mixed solution | 0.2-0.6 | mM |
| Mg2+ | 2-8 | mM |
| SEPT9 forward primer | 0.1-2 | μM |
| SEPT9 reverse primer | 0.1-2 | μM |
| SEPT9 blocker | 0.1-1.5 | μM |
| SEPT9 probe | 0.01-0.2 | μM |
| SDC2 forward primer | 0.1-1 | μM |
| SDC2 reverse primer | 0.1-1 | μM |
| SDC2 blocker | 0.1-1 | μM |
| SDC2 probe | 0.01-0.2 | μM |
| ACTB forward primer | 0.2 | μM |
| ACTB reverse primer | 0.2 | μM |
| ACTB probe | 0.1 | μM |
| Deionized water | Add deionized water to 15 μL | |
| DNA template | 15 | μL |

The present invention further provides an application of the primer and probe set for detecting methylation of SEPT9 and SDC2 in a biological sample as a reagent in early screening/detection/diagnosis of colorectal cancer and precancerous lesions thereof, and further provides an application of the amplification system as a reagent in early screening/detection/diagnosis of colorectal cancer and precancerous lesions thereof.

Compared with the prior art, the present invention has the beneficial effect of simultaneously detecting methylation levels of SEPT9 and SDC2 with high sensitivity and specificity, for use in screening/detection/diagnosis of early-stage colorectal cancer.

### BRIEF DESCRIPTION OF THE DRAWINGS

FIG. 1 is an amplification plot of 1000 copies, 100 copies, and 10 copies of methylated SEPT9 genes;

FIG. 2 is an amplification plot of 1000 copies, 100 copies, and 10 copies of methylated SDC2 genes;

FIG. 3 is a diagram showing the effects of a single SEPT9 primer probe set and a combined primer probe set of SEPT9 and SDC2 on detecting methylated SEPT9;

FIG. 4 is a diagram showing the effects of a single SDC2 primer probe set and a combined primer probe set of SEPT9 and SDC2 on detecting methylated SDC2;

FIG. 5 is a diagram showing the effects of a combined primer probe set of SEPT9 and SDC2 on detecting 17 cases of colorectal cancer tissues and corresponding cancer-adjacent tissues.

### DETAILED DESCRIPTION

The present invention would be further described in detail with reference to the following embodiments, so that a person skilled in the art can implement the present invention with reference to the description text.

### Embodiment 1

A genomic sequence of a cell line whose SEPT9 and SDC2 genes are known to be fully methylated is taken as a DNA template; the DNA template is converted by heating at 70° C. for 60 min in an ammonium bisulfite solution of 8 M, and then purified by an Axygen gel purification kit to obtain the converted DNA template which is then subjected to PCR reaction. The PCR reaction system is shown in Table 1, and the PCR reaction conditions are shown in Table 2.

TABLE 1
PCR reaction system in embodiment 1

| Components | Final concentration | |
|---|---|---|
| PCR reaction buffer | 1 | X |
| DNA polymerase | 0.1 | U/μL |
| dNTPs mixed solution | 0.4 | mM |
| Mg2+ | 2 | mM |
| SEQ ID NO: 20 | 0.5 | μM |
| SEQ ID NO: 38 | 0.5 | μM |
| SEQ ID NO: 53 | 0.4 | μM |
| SEQ ID NO: 79 | 0.1 | μM |
| SEQ ID NO: 99 | 0.2 | μM |
| SEQ ID NO: 127 | 0.2 | μM |
| SEQ ID NO: 144 | 0.1 | μM |
| SEQ ID NO: 167 | 0.1 | μM |
| SEQ ID NO: 175 | 0.2 | μM |
| SEQ ID NO: 176 | 0.2 | μM |
| SEQ ID NO: 177 | 0.1 | μM |
| Deionized water | Add deionized water to 15 μL | |
| Converted DNA template | 15 | μL |

3′-ends of SEQ ID NO: 53 and SEQ ID NO: 144 are modified using a phosphate group and C3, respectively. The reaction system consists of 15 μL of a reaction solution and 15 μL of the converted DNA template, with a total volume of 30 μL. 15 μL of the reaction solution is the remaining components except the converted DNA template. The following is the same.

TABLE 2
PCR reaction conditions

| Number | | | |
|---|---|---|---|
| of cycles | Temperature | Duration | |
| 1 | 95° C. | 30 | min |
| 50 | 95° C. | 10 | s |
| | 56° C. | 30 | s |
| 1 | 40° C. | 30 | s |

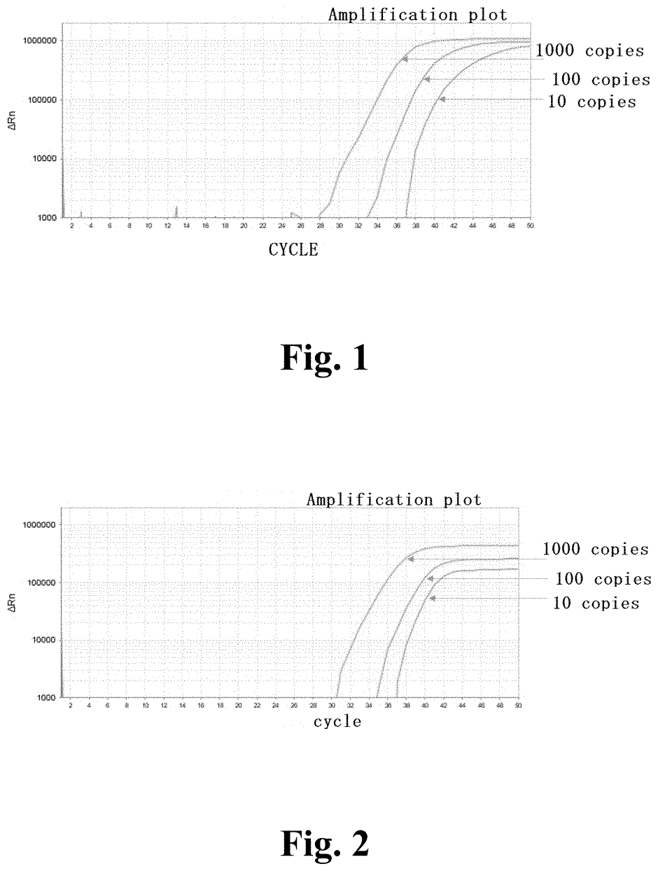

FIG. 1 shows amplification curves of 1000 copies, 100 copies, and 10 copies of methylated SEPT9 genes in embodiment 1. FIG. 2 shows amplification curves of 1000 copies, 100 copies, and 10 copies of methylated SDC2 genes in embodiment 1. As shown in FIGS. 1 and 2, the methylated SEPT9 and SDC2 have good amplification effects under the same reaction conditions, and the detection sensitivity reaches 10 copies.

### Embodiment 2

A genomic sequence of a cell line whose SEPT9 and SDC2 genes are known to be fully methylated is taken as a DNA template; the DNA template is converted by heating at 80° C. for 40 min in an ammonium bisulfite solution of 9 M, and then purified by an Axygen gel purification kit to obtain the converted DNA template which is then subjected to PCR reaction. The components of the PCR reaction using the SEPT9 primer probe set alone are shown in Table 3. The components of the PCR reaction using the SDC2 primer probe set alone are shown in Table 4. The components of the PCR reaction using the combined primer probe set of SDC2 and SEPT9 are shown in Table 5. The PCR reaction conditions in embodiment 2 are shown in Table 6.

TABLE 3
PCR components using SEPT9 primer probe set alone

| Components | Final concentration | |
|---|---|---|
| PCR reaction buffer | 1 | X |
| DNA polymerase | 0.1 | U/μL |
| dNTPs mixed solution | 0.4 | mM |
| Mg2+ | 2 | mM |
| SEQ ID NO: 3 | 1 | μM |
| SEQ ID NO: 28 | 1 | μM |
| SEQ ID NO: 52 | 0.8 | μM |
| SEQ ID NO: 72 | 0.1 | μM |
| SEQ ID NO: 175 | 0.2 | μM |
| SEQ ID NO: 176 | 0.2 | μM |
| SEQ ID NO: 177 | 0.1 | μM |
| Deionized water | Add deionized water to 15 μL | |
| Converted DNA template | 15 | μL |

A 3′-end of SEQ ID NO: 52 is modified using C9.

TABLE 4
PCR components using SDC2 primer probe set alone

| Components | Final concentration | |
|---|---|---|
| PCR reaction buffer | 1 | X |
| DNA polymerase | 0.1 | U/μL |
| dNTPs mixed solution | 0.4 | mM |
| Mg2+ | 2 | mM |
| SEQ ID NO: 91 | 0.4 | μM |
| SEQ ID NO: 118 | 0.4 | μM |
| SEQ ID NO: 144 | 0.2 | μM |
| SEQ ID NO: 164 | 0.2 | μM |
| SEQ ID NO: 175 | 0.2 | μM |
| SEQ ID NO: 176 | 0.2 | μM |
| SEQ ID NO: 177 | 0.1 | μM |
| Deionized water | Add deionized water to 15 μL | |
| Converted DNA template | 15 | μL |

A 3′-end of SEQ ID NO: 144 is modified using C9.

TABLE 5
PCR components using a combined primer
probe set of SEPT9 and SDC2

| Components | Final concentration | |
|---|---|---|
| PCR reaction buffer | 1 | X |
| DNA polymerase | 0.1 | U/μL |
| dNTPs mixed solution | 0.4 | mM |
| Mg2+ | 2 | mM |
| SEQ ID NO: 3 | 1 | μM |
| SEQ ID NO: 28 | 1 | μM |
| SEQ ID NO: 52 | 0.8 | μM |
| SEQ ID NO: 72 | 0.1 | μM |
| SEQ ID NO: 91 | 0.4 | μM |
| SEQ ID NO: 118 | 0.4 | μM |
| SEQ ID NO: 144 | 0.2 | μM |
| SEQ ID NO: 164 | 0.2 | μM |
| SEQ ID NO: 175 | 0.2 | μM |
| SEQ ID NO: 176 | 0.2 | μM |
| SEQ ID NO: 177 | 0.1 | μM |
| Deionized water | Add deionized water to 15 μL | |
| Converted DNA template | 15 | μL |

3′-ends of SEQ ID NO: 52 and SEQ ID NO: 144 are modified using C9.

TABLE 6
PCR reaction conditions in embodiment 2

| Number | | | |
|---|---|---|---|
| of cycles | Temperature | Duration | |
| 1 | 95° C. | 30 | min |
| 50 | 95° C. | 10 | s |
| | 56° C. | 30 | s |
| 1 | 40° C. | 30 | s |

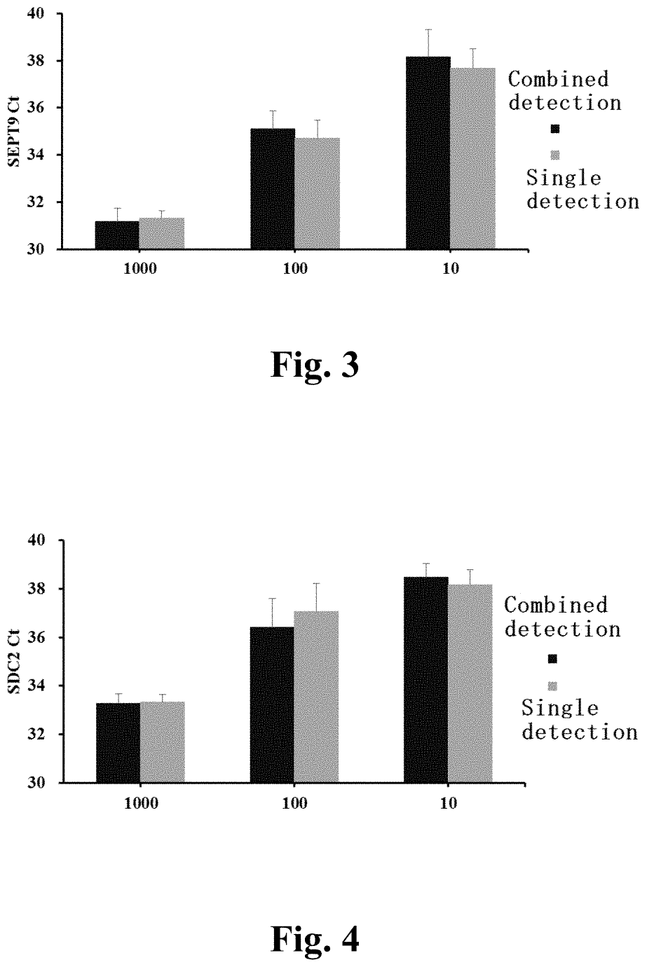

FIG. 3 is a schematic diagram showing the effects of a single SEPT9 primer probe set and a combined primer probe set of SEPT9 and SDC2 on detecting methylated SEPT9. FIG. 4 is a schematic diagram showing the effects of a single SDC2 primer probe set and a combined primer probe set of SEPT9 and SDC2 on detecting methylated SDC2. As can be seen in FIGS. 3 and 4, there is no significant difference between combined detection and separate detection of SDC2 and SEPT9.

### Embodiment 3

Colorectal cancer tissues and matched cancer-adjacent tissues are taken as detection objects. After DNA is extracted using a Qiagen DNeasy Blood & Tissue Kit, an ammonium bisulfite solution of 6 M, sodium bisulfite of 3 M, and anhydrous sodium sulfate of 1 M are used to be prepared into a bisulfite solution of 10 M. After DNA is converted, and then purified by an Axygen gel purification kit, the converted DNA template is obtained and then subjected to PCR reaction. The conversion condition is heating at 90° C. for 30 min. The PCR reaction system is shown in Table 7, and the PCR reaction conditions are shown in Table 8.

TABLE 7
PCR components in embodiment 3

| Components | Final concentration | |
|---|---|---|
| PCR reaction buffer | 1 | X |
| DNA polymerase | 0.1 | U/μL |
| dNTPs mixed solution | 0.4 | mM |
| Mg2+ | 2 | mM |
| SEQ ID NO: 1 | 1.5 | μM |
| SEQ ID NO: 30 | 1.5 | μM |
| SEQ ID NO: 55 | 1 | μM |
| SEQ ID NO: 80 | 0.2 | μM |
| SEQ ID NO: 96 | 0.5 | μM |
| SEQ ID NO: 129 | 0.5 | μM |
| SEQ ID NO: 156 | 0.2 | μM |
| SEQ ID NO: 163 | 0.1 | μM |
| SEQ ID NO: 175 | 0.2 | μM |
| SEQ ID NO: 176 | 0.2 | μM |
| SEQ ID NO: 177 | 0.1 | μM |
| Deionized water | Add deionized water to 15 μL | |
| Converted DNA template | 15 | μL |

3′-ends of SEQ ID NO: 55 and SEQ ID NO: 156 are modified using a phosphate group and C18, respectively.

TABLE 8
PCR reaction conditions in embodiment 3

| Number | | | |
|---|---|---|---|
| of cycles | Temperature | Duration | |
| 1 | 95° C. | 30 | min |
| 50 | 95° C. | 10 | s |
| | 56° C. | 30 | s |
| 1 | 40° C. | 30 | s |

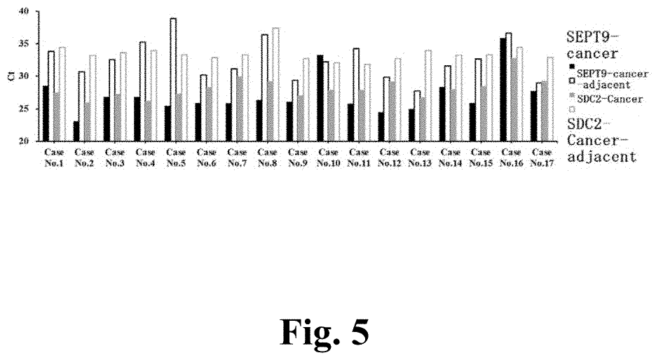

FIG. 5 is a schematic diagram showing the effects of a combined primer probe set of SEPT9 and SDC2 on detecting 17 cases of colorectal cancer tissues and corresponding cancer-adjacent tissues. It can be seen from FIG. 5 that both SEPT9 and SDC2 can well distinguish colorectal cancer tissues from cancer-adjacent tissues, and therefore, SEPT9 and SDC2 can be used for early screening/detection/diagnosis of colorectal cancer.

### Embodiment 4

20 cases of colorectal serum samples of stages II-IV are collected with a volume of about 1.5 mL, labeled as a colorectal cancer group, and 56 cases of serum samples from patients without significant gastrointestinal diseases are collected with a volume of about 1 mL, labeled as a normal group. DNA is extracted by a cell-free DNA extraction kit of Suzhou VersaBio Technologies, Inc., then heated at 80° C. for 45 min for conversion in an ammonium bisulfite solution of 9 M, and then purified by an Axygen gel purification kit to obtain a converted DNA template which is then subjected to PCR reaction. The PCR reaction system in embodiment 4 is shown in Table 9, and the reaction conditions are shown in Table 10. The effects of a combined primer probe set of SEPT9 and SDC2 on detecting SEPT9 and SDC2 methylation in cell-free DNA are shown in Table 11.

TABLE 9
PCR components in embodiment 4

| Components | Final concentration | |
|---|---|---|
| PCR reaction buffer | 1 | X |
| DNA polymerase | 0.1 | U/μL |
| dNTPs mixed solution | 0.4 | mM |
| Mg2+ | 2 | mM |
| SEQ ID NO: 13 | 1.5 | μM |
| SEQ ID NO: 50 | 1.5 | μM |
| SEQ ID NO: 61 | 1 | μM |
| SEQ ID NO: 74 | 0.2 | μM |
| SEQ ID NO: 97 | 0.4 | μM |
| SEQ ID NO: 125 | 0.4 | μM |
| SEQ ID NO: 155 | 0.2 | μM |
| SEQ ID NO: 165 | 0.1 | μM |
| SEQ ID NO: 175 | 0.2 | μM |
| SEQ ID NO: 176 | 0.2 | μM |
| SEQ ID NO: 177 | 0.1 | μM |
| Deionized water | Add deionized water to 15 μL | |
| DNA template | 15 | μL |

3′-ends of SEQ ID NO: 61 and SEQ ID NO: 155 are modified using C3.

TABLE 10
PCR reaction conditions in embodiment 4

| Number | | | |
|---|---|---|---|
| of cycles | Temperature | Duration | |
| 1 | 95° C. | 30 | mm |
| 50 | 95° C. | 10 | s |
| | 56° C. | 30 | s |
| 1 | 40° C. | 30 | s |

TABLE 11
Effects of a combined primer probe set of SEPT9 and SDC2 in detecting
SEPT9 and SDC2 methylation in cell-free DNA

| | Detected | Number | | | |
|---|---|---|---|---|---|
| Components | gene | of cases | Positive | Sensitivity | Specificity |
| Colorectal | SEPT9 | 20 | 15 | 75% | / |
| cancer | SDC2 | 20 | 18 | 90% | / |
| group | SEPT9 + | 20 | 20 | 100% | / |
| | SDC2 | | | | |
| Normal | SEPT9 | 56 | 55 | / | 98% |
| group | SDC2 | 56 | 51 | / | 91% |
| | SEPT9 + | 56 | 50 | / | 89% |
| | SDC2 | | | | |

It can be seen from Table 11 that the combined detection of SEPT9 and SDC2 can significantly improve the sensitivity of colorectal cancer, and still has high specificity.

Sequences 1-177 of the present invention are shown in Table 12 below.

TABLE 12
Primer and probe sequences

| Remark | SED ID | Sequences |
|---|---|---|
| SEPT9 | SEQ ID NO: 1 | AAATAATCCCATCCAACTAC |
| forward | SED ID NO: 2 | GTAGTTGGATGGGATTATTT |
| primer | SED ID NO: 3 | AAATAATCCCATCCAACTACG |
| | SED ID NO: 4 | CGTAGTTGGATGGGATTATTT |
| | SED ID NO: 5 | AATAATCCCATCCAACTAC |
| | SED ID NO: 6 | GTAGTTGGATGGGATTATT |
| | SED ID NO: 7 | AATAATCCCATCCAACTACG |
| | SED ID NO: 8 | CGTAGTTGGATGGGATTATT |
| | SED ID NO: 9 | ATAATCCCATCCAACTACG |
| | SED ID NO: 10 | CGTAGTTGGATGGGATTAT |
| | SED ID NO: 11 | RAAATAATCCCATCCAACTA |
| | SED ID NO: 12 | CRAAATAATCCCATCCAACTA |
| | SED ID NO: 13 | TAGTTGGATGGGATTATTTYG |
| | SED ID NO: 14 | RAAATAATCCCATCCAACT |
| | SED ID NO: 15 | AGTTGGATGGGATTATTTY |
| | SED ID NO: 16 | CRAAATAATCCCATCCAACT |
| | SED ID NO: 17 | AGTTGGATGGGATTATTTYG |
| | SED ID NO: 18 | CRAAATAATCCCATCCAAC |
| | SED ID NO: 19 | GTTGGATGGGATTATTTYG |
| | SED ID NO: 20 | CCACCTTCGAAATCCGAAAT |
| | SED ID NO: 21 | ATTTCGGATTTCGAAGGTGG |
| | SED ID NO: 22 | ATCCGAAATAATCCCATCCAA |
| | SED ID NO: 23 | TTGGATGGGATTATTTCGGAT |
| | SED ID NO: 24 | GATTTCGAAGGTGGGTGTTGGG |
| | SED ID NO: 25 | CCCAACACCCACCTTCGAAATC |
| SEPT9 | SED ID NO: 26 | TCGTCGTTGTTTTTCGCGCGA |
| reverse | SED ID NO: 27 | TCGCGCGAAAAACAACGACGA |
| primer | SED ID NO: 28 | CATAATAACTAATAAACAACRAATC |
| | SED ID NO: 29 | GATTYGTTGTTTATTAGTTATTATG |
| | SED ID NO: 30 | GATTYGTTGTTTATTAGTTATTAT |
| | SED ID NO: 31 | ATAATAACTAATAAACAACRAATC |
| | SED ID NO: 32 | GTTGTTTATTAGTTATTATGT |
| | SED ID NO: 33 | ACATAATAACTAATAAACAAC |
| | SED ID NO: 34 | GTAGGGTTCGGGTTTC |
| | SED ID NO: 35 | GAAACCCGAACCCTAC |
| | SED ID NO: 36 | TTCGTCGCTGTTTTTC |
| | SED ID NO: 37 | GAAAAACAGCGACGAA |
| | SED ID NO: 38 | GCGCGATTCGTTGTTTATTA |
| | SED ID NO: 39 | TAATAAACAACGAATCGCGC |
| | SED ID NO: 40 | TAGGGTTCGGGTTTCGT |
| | SED ID NO: 41 | ACGAAACCCGAACCCTA |
| | SED ID NO: 42 | GTAGGGTTCGGGTTTY |
| | SED ID NO: 43 | RAAACCCGAACCCTAC |
| | SED ID NO: 44 | TTYGTYGCTGTTTTTY |
| | SED ID NO: 45 | RAAAAACAGCRACRAA |
| | SED ID NO: 46 | GYGYGATTYGTTGTTTATTA |
| | SED ID NO: 47 | TAATAAACAACRAATCRCRC |
| | SED ID NO: 48 | TAGGGTTYGGGTTTYGT |
| | SED ID NO: 49 | ACRAAACCCRAACCCTA |
| | SED ID NO: 50 | YGATTYGTTGTTTATTAGTTATTATG |
| | SED ID NO: 51 | CATAATAACTAATAAACAACRAATCR |
| SEPT9 | SED ID NO: 52 | TTGGATTTTGTGGTTAATGTGTAG |
| blocker | SED ID NO: 53 | TGTTGGATTTTGTGGTTAATGTGTAG |
| | SED ID NO: 54 | GTTATTATGTTGGATTTTGTG |
| | SED ID NO: 55 | GTTATTATGTTGGATTTTGTGGTTAA |
| | | TGTGTA |
| | SED ID NO: 56 | GTTATTATGTTGGATTTTGTGGTTAA |
| | | TGTGT |
| | SED ID NO: 57 | GTTATTATGTTGGATTTTGTGGTTAA |
| | | TGTG |
| | SED ID NO: 58 | GTTATTATGTTGGATTTTGTGGT |
| | SED ID NO: 59 | GTTATTATGTTGGATTTTGTGGTTAA |
| | | TGTGTAGAAAA |
| | SED ID NO: 60 | TGTTTATTAGTTATTATGTTGGATTT |
| | | TGTGGT |
| | SED ID NO: 61 | TTATTATGTTGGATTTTGTGGTTAAT |
| | | GTGTAGAAA |
| | SED ID NO: 62 | TTTATTAGTTATTATGTTGGATTTTG |
| | | TGGT |
| | SED ID NO: 63 | TTATTAGTTATTATGTTGGATTTTGT |
| | | GGT |
| | SED ID NO: 64 | TATTAGTTATTATGTTGGATTTTGTG |
| | | GT |
| | SED ID NO: 65 | ATTAGTTATTATGTTGGATTTTGTGGT |
| | SED ID NO: 66 | TTAGTTATTATGTTGGATTTTGTGGT |
| | SED ID NO: 67 | TAGTTATTATGTTGGATTTTGTGGT |
| SEPT9 | SED ID NO: 68 | TTCGCGGTTAACGCGTAG |
| probe | SED ID NO: 69 | CTACGCGTTAACCGCGAA |
| | SED ID NO: 70 | ATTTCGCGGTTAACGCGT |
| | SED ID NO: 71 | ACGCGTTAACCGCGAAAT |
| | SED ID NO: 72 | GTTAACCGCGAAATCCGA |
| | SED ID NO: 73 | TCGGATTTCGCGGTTAAC |
| | SED ID NO: 74 | CGTTAACCGCGAAATCCGA |
| | SED ID NO: 75 | TCGGATTTCGCGGTTAACG |
| | SED ID NO: 76 | GCGTTAACCGCGAAATCCGA |
| | SED ID NO: 77 | TCGGATTTCGCGGTTAACGC |
| | SED ID NO: 78 | CGCGTTAACCGCGAAATCCGA |
| | SED ID NO: 79 | CGTTAACCGCGAAATCCG |
| | SED ID NO: 80 | GCGTTAACCGCGAAATCCGA |
| | SED ID NO: 81 | CGCGTTAACCGCGAAATCCGA |
| | SED ID NO: 82 | GTCGGATTTCGCGGTTAA |
| | SED ID NO: 83 | GTCGGATTTCGCGGTTAAC |
| | SED ID NO: 84 | GTCGGATTTCGCGGTTAACG |
| | SED ID NO: 85 | GTCGGATTTCGCGGTTAACGC |
| | SED ID NO: 86 | GTCGGATTTCGCGGTTAACGCG |
| | SED ID NO: 87 | TCGGATTTCGCGGTTAAC |
| | SED ID NO: 88 | CGGATTTCGCGGTTAACG |
| | SED ID NO: 89 | CGGATTTCGCGGTTAACGC |
| | SED ID NO: 90 | CGGATTTCGCGGTTAACGCG |
| SDC2 | SED ID NO: 91 | AGTAAGAAGAGTTTTAGAGAGT |
| forward | SED ID NO: 92 | ACTCTCTAAAACTCTTCTTACT |
| primer | SED ID NO: 93 | GAAGAGTTTTAGAGAGTAGTT |
| | SED ID NO: 94 | AACTACTCTCTAAAACTCTTC |
| | SED ID NO: 95 | CGCGTTTTCGGGGCGTAG |
| | SED ID NO: 96 | CTACGCCCCGAAAACGCG |
| | SED ID NO: 97 | GGAGGAAGCGAGYGTTTT |
| | SED ID NO: 98 | AAAACRCTCGCTTCCTCC |
| | SED ID NO: 99 | TAGAAATTAATAAGTGAGAGG |
| | SED ID NO: 100 | CCTCTCACTTATTAATTTCTA |
| | SED ID NO: 101 | TTCGGGGCGTAGTTGCG |
| | SED ID NO: 102 | CGCAACTACGCCCCGAA |
| | SED ID NO: 103 | GCGTAGGAGGAGGAAGCGAGC |
| | SED ID NO: 104 | GCTCGCTTCCTCCTCCTACGC |
| | SED ID NO: 105 | GYGTAGGAGGAGGAAGCGAGC |
| | SED ID NO: 106 | GCTCGCTTCCTCCTCCTACRC |
| | SED ID NO: 107 | GYGTAGGAGGAGGAAGYGAGC |
| | SED ID NO: 108 | GCTCRCTTCCTCCTCCTACRC |
| | SED ID NO: 109 | GYGTAGGAGGAGGAAGYGAGY |
| | SED ID NO: 110 | RCTCRCTTCCTCCTCCTACRC |
| | SED ID NO: 111 | CGTAGGAGGAGGAAGCGA |
| | SED ID NO: 112 | TCGCTTCCTCCTCCTACG |
| | SED ID NO: 113 | YGTAGGAGGAGGAAGYGA |
| | SED ID NO: 114 | TCRCTTCCTCCTCCTACR |
| | SED ID NO: 115 | AATAAGTGAGAGGGCGTC |
| | SED ID NO: 116 | GACGCCCTCTCACTTATT |
| | SED ID NO: 117 | TTAATAAGTGAGAGGGTCG |
| SDC2 | SED ID NO: 118 | GTTTCGGATTCGTGTGCGC |
| reverse | SED ID NO: 119 | GCGCACACGAATCCGAAAC |
| primer | SED ID NO: 120 | CGGATTCGTGTGCGCGGGTT |
| | SED ID NO: 121 | AACCCGCGCACACGAATCCG |
| | SED ID NO: 122 | GCGGTATTTTGTTTCGGATTCGT |
| | SED ID NO: 123 | ACGAATCCGAAACAAAATACCGC |
| | SED ID NO: 124 | CGTAATCGTTGCGGTATTT |
| | SED ID NO: 125 | AAATACCGCAACGATTACG |
| | SED ID NO: 126 | TTCGAGTTTTCGAGTTTGAG |
| | SED ID NO: 127 | CTCAAACTCGAAAACTCGAA |
| | SED ID NO: 128 | CGTAATCGTTGCRGTATTT |
| | SED ID NO: 129 | AAATACYGCAACGATTACG |
| | SED ID NO: 130 | CGTAATCRTTGCRGTATTT |
| | SED ID NO: 131 | AAATACYGCAAYGATTACG |
| | SED ID NO: 132 | CRTAATCRTTGCRGTATTT |
| | SED ID NO: 133 | AAATACYGCAAYGATTAYG |
| | SED ID NO: 134 | TTCGAGTTTTCRAGTTTGAG |
| | SED ID NO: 135 | CTCAAACTYGAAAACTCGAA |
| | SED ID NO: 136 | TTCRAGTTTTCRAGTTTGAG |
| | SED ID NO: 137 | CTCAAACTYGAAAACTYGAA |
| | SED ID NO: 138 | CGAGTTCGAGTTTTCGAGTTTG |
| | SED ID NO: 139 | CAAACTCGAAAACTCGAACTCG |
| | SED ID NO: 140 | CGAGTTCGAGTTTTCGAGTTT |
| | SED ID NO: 141 | AAACTCGAAAACTCGAACTCG |
| | SED ID NO: 142 | CGAGTTCGAGTTTTCGAGTT |
| | SED ID NO: 143 | AACTCGAAAACTCGAACTCG |
| SDC2 | SED ID NO: 144 | GTAGGTGTAGGAGGAGGAAGTGAG |
| blocker | SED ID NO: 145 | CTCACTTCCTCCTCCTACACCTAC |
| | SED ID NO: 146 | TTTGGGGTGTAGTTGTGGGTGG |
| | SED ID NO: 147 | CCACCCACAACTACACCCCAAA |
| | SED ID NO: 148 | TGAGTTTGAGTTTTTGAGTTTG |
| | SED ID NO: 149 | CAAACTCAAAAACTCAAACTCA |
| | SED ID NO: 150 | TTTGAGTTTGAGTTTTTGAG |
| | SED ID NO: 151 | CTCAAAAACTCAAACTCAAA |
| | SED ID NO: 152 | GGCGTAGTTGCGGGCGGCGG |
| | SED ID NO: 153 | CCGCCGCCCGCAACTACGCC |
| | SED ID NO: 154 | TGAGTGTTTTTGAGTTTTGAGTTTG |
| | SED ID NO: 155 | AACTTGAAAACTTGAACTTGAAACT |
| | SED ID NO: 156 | TGAGTGTTTTTGAGTTTTGAGTTTGAG |
| | | TTTTTG |
| SDC2 | SED ID NO: 157 | GGCGTAGGAGGAGGAAGCGAGCG |
| probe | SED ID NO: 158 | CGCTCGCTTCCTCCTCCTACGCC |
| | SED ID NO: 159 | GGCGTAGTTGCGGGCGGC |
| | SED ID NO: 160 | GCCGCCCGCAACTACGCC |
| | SED ID NO: 161 | TAGTTGCGGGCGGCGGG |
| | SED ID NO: 162 | CCCGCCGCCCGCAACTA |
| | SED ID NO: 163 | AGTTCGAGTTTTCGAGTTTGAG |
| | SED ID NO: 164 | AACTCGAAAACTCGAACTCGAAACT |
| | SED ID NO: 165 | AGTTTCGAGTTCGAGTTTTCGAGTT |
| | SED ID NO: 166 | CTCAAACTCGAAAACTCGAACT |
| | SED ID NO: 167 | GCGTAGGAGGAGGAAGCGA |
| | SED ID NO: 168 | CGGGCGGCGGGAGTAGGCGT |
| | SED ID NO: 169 | TCGCTTCCTCCTCCTACGC |
| | SED ID NO: 170 | ACGCCTACTCCCGCCGCCCG |
| | SED ID NO: 171 | CGTAGGAGGAGGAAGCGAGC |
| | SED ID NO: 172 | GCTCGCTTCCTCCTCCTACG |
| | SED ID NO: 173 | CGAGTTCGAGTTTTCGAGTTTGAG |
| | SED ID NO: 174 | CTCAAACTCGAAAACTCGAACTCG |
| ACTB | SED ID NO: 175 | GTGATGGAGGAGGTTTAGTAAGT |
| forward | | |
| primer | | |
| ACTB | SED ID NO: 176 | CCAATAAAACCTACTCCTCCCTTA |
| reverse | | |
| primer | | |
| ACTB | SED ID NO: 177 | CCACCACCCAACACACAATAACAAAC |
| probe | | AC |

Although the embodiments of the present invention have been disclosed as above, they are not limited merely to those set forth in the description and the embodiments, and they may be applied to various fields suitable for the present invention. For a person skilled in the art, other modifications may be easily achieved without departing from the general concept defined by the claims and their equivalents, and the present invention is not limited to the particular details.

1. A primer and probe set for diagnosis, detection or screening of colorectal cancer, comprising amplification primers, a blocker primer, and a probe of SEPT9, and amplification primers, a blocker primer, and a probe of SDC2.

2. The primer and probe set for diagnosis, detection or screening of colorectal cancer according to claim 1, wherein,
a forward primer of SEPT9 is selected from any one of SEQ ID NOs: 1-25, a reverse primer of SEPT9 is selected from any one of SEQ ID NOs: 26-51, the blocker primer of SEPT9 is selected from any one of SEQ ID NOs: 52-67, and the probe of SEPT9 is selected from any one of SEQ ID NOs: 68-90.

3. The primer and probe set for diagnosis, detection or screening of colorectal cancer according to claim 1 or 2, wherein,
a forward primer of SDC2 is selected from any one of SEQ ID NOs: 91-117, a reverse primer of SDC2 is selected from any one of SEQ ID NOs: 118-143, the blocker primer of SDC2 is selected from any one of SEQ ID NOs: 144-156, and the probe of SDC2 is selected from any one of SEQ ID NOs: 157-174.

4. The primer and probe set for diagnosis, detection or screening of colorectal cancer according to claim 1, further comprising a reference gene primer probe set, wherein the reference gene is ACTB, a forward primer of ACTB is SEQ ID NO: 175, a reverse primer of ACTB is SEQ ID NO: 176, and a probe of ACTB is selected from SEQ ID NO: 177.

5. The primer and probe set for diagnosis, detection or screening of colorectal cancer according to claim 1, wherein,
a detection object is a DNA sample obtained by extracting and purifying genomic DNA from a biological sample and then converting same using bisulfite; the bisulfite is one or more of ammonium bisulfite, sodium bisulfite, and anhydrous sodium sulfate, and the concentration of the bisulfite is 8-10 M; the reaction conditions of the bisulfite conversion process are that DNA is added into a bisulfite solution of 8-10 M, and heated at 70-90° C. for 30-60 min.

6. The primer and probe set for diagnosis, detection or screening of colorectal cancer according to claim 1, wherein,
detection of SEPT9 methylation, SDC2 methylation, and ACTB is performed simultaneously in one quantitative fluorescent PCR reaction, or a quantitative fluorescent PCR reaction is performed separately for one site.

7. The primer and probe set for diagnosis, detection or screening of colorectal cancer according to claim 1, wherein,
the blocker primer of SEPT9 and/or the blocker primer of SDC2 are modified using one of a phosphate group, C3, C9, and C18 at 3′-ends.

8. An amplification system comprising the primer and probe set for diagnosis, detection or screening of colorectal cancer according to claim 1, wherein,
specific components of the amplification system are:

| Components | Final concentration | |
|---|---|---|
| PCR reaction buffer | 1 | X |
| DNA polymerase | 0.08-0.2 | U/μL |
| dNTPs mixed solution | 0.2-0.6 | mM |
| Mg2+ | 2-8 | mM |
| SEPT9 forward primer | 0.1-2 | μM |
| SEPT9 reverse primer | 0.1-2 | μM |
| SEPT9 blocker | 0.1-1.5 | μM |
| SEPT9 probe | 0.01-0.2 | μM |
| SDC2 forward primer | 0.1-1 | μM |
| SDC2 reverse primer | 0.1-1 | μM |
| SDC2 blocker | 0.1-1 | μM |
| SDC2 probe | 0.01-0.2 | μM |
| ACTB forward primer | 0.2 | μM |
| ACTB reverse primer | 0.2 | μM |
| ACTB probe | 0.1 | μM |
| Deionized water | Add deionized water to 15 μL | |
| DNA template | 15 | μL |

9. A method of the primer and probe set for diagnosis, detection or screening of colorectal cancer according to claim 1, comprising applying as a reagent in early screening/detection/diagnosis of colorectal cancer and precancerous lesions thereof.

10. (canceled)

11. The primer and probe set for diagnosis, detection or screening of colorectal cancer according to claim 2, wherein,
a forward primer of SDC2 is selected from any one of SEQ ID NOs: 91-117, a reverse primer of SDC2 is selected from any one of SEQ ID NOs: 118-143, the blocker primer of SDC2 is selected from any one of SEQ ID NOs: 144-156, and the probe of SDC2 is selected from any one of SEQ ID NOs: 157-174.

12. The primer and probe set for diagnosis, detection or screening of colorectal cancer according to claim 2, wherein,
the blocker primer of SEPT9 and/or the blocker primer of SDC2 are modified using one of a phosphate group, C3, C9, and C18 at 3′-ends.

13. The primer and probe set for diagnosis, detection or screening of colorectal cancer according to claim 3, wherein,
the blocker primer of SEPT9 and/or the blocker primer of SDC2 are modified using one of a phosphate group, C3, C9, and C18 at 3′-ends.
